(19) United States
(12) Patent Application Publication
(10) Pub. No.: US 2022/0249190 A1
(43) Pub. Date: Aug. 11, 2022

(54) SKULL PORTAL DEVICE FOR CRANIAL ACCESS

(71) Applicant: SURGITECH LTD., Vancouver (CA)

(72) Inventors: John KELLY, Vancouver (CA); Frank FLETCHER, Vancouver (CA); James ARTINDALE, Vancouver (CA)

(21) Appl. No.: 17/618,614

(22) PCT Filed: Jun. 12, 2020
(86) PCT No.: PCT/CA2020/050813

§ 371 Date: Dec. 13, 2021

Related U.S. Application Data
(60) Provisional application No. 62/861,071, filed on Jun. 13, 2019.

(51) Int. Cl. A61B 90/11

(52) U.S. Cl. CPC A61B 90/11; A61B 10/0233

(57) ABSTRACT

Apparatus for providing repeated cranial access comprises a hollow shaft sized to fit within a hole extending through a skull bone, the hollow shaft having a length of at least a thickness of the skull bone, a lip extending laterally form a first end of the hollow shaft for abutting the skull bone around the hole, and, a plug sized to fit into the hollow shaft, the plug extending at least the length of the hollow shaft and configured to be removably secured within the hollow shaft.

### TECHNICAL FIELD

The present disclosure relates to an apparatus and method for closing the skull after cranial surgery and providing access to the brain post-surgery.

### BACKGROUND

In order to perform certain types of surgeries, such as brain surgery, access must be achieved by opening the skull (or cranium) in order to permit entry to the brain or surrounding intracranial spaces (epidural and subdural). A skull opening in its most simple form is called a burr hole. A bigger opening of the skull, a craniotomy, involves temporarily removing a window of bone called a craniotomy bone flap. The craniotomy bone flap is replaced and secured to the skull at the end of the procedure.

In order to perform a craniotomy, in an embodiment, one or more burrs hole are typically required. Often multiple burr holes are placed then interconnected by sawing through the skull to create the bone flap.

Once the skull is opened the underlying dura matter is incised providing access to the brain. Brain surgery including tumor biopsy and/or removal, hemorrhage evacuation, shunt insertion, deep brain stimulation (DBS) and other related procedures involving implantation of leads and catheters are increasingly used to treat cancers, intracranial and intracerebral hemorrhages, brain aneurysms, Parkinson's disease, seizure disorders, and numerous other debilitating diseases. During these procedures, one or more burr holes are typically made. Blood or tissue can be removed through a burr hole or larger cranial opening. In addition, sometimes a catheter, lead, or other portion of medical device is strategically placed through a burr hole transiently or indefinitely at a target site in the brain. When leaving a portion of a medical device inserted in the brain, the medical device must be secured where it exits the skull so as to prevent movement of the device from the precise target site in the brain. When performing a procedure through a burr hole or small opening, such as a procedure using an endoscope, there is sometimes a need to fix the device in order to prevent inadvertent injury to the brain. Once a craniotomy is completed the bone must be reaffixed to the skull. Burr holes can either be left open, can be covered or can be filled.

Certain currently available burr hole plugs are used to hold individual exiting devices in place, but often do so inadequately. During placement of current burr hole plugs, the exiting device may move from its precise target site in the brain.

In some instances, if a surgeon needs to re-enter the brain post-surgery, the surgeon may typically need to reopen a previous burr hole site or entire craniotomy incision and site in order to access the intracranial cavity and brain. Re-entry may be desirable, for example, when a tumor is suspected to be re-growing in a previous resection cavity and resampling or another removal is required, when a subdural or intracerebral hematoma has re-accumulated and needs re-evacuation, when an intracranial monitoring device requires replacement, or when a procedure to establish Cerebro Spinal Fluid (CSF) flow, such as endoscopic ventriculostomy, needs to be repeated. Reopening previous burr holes or entire craniotomy sites requires long and difficult procedures with an increased chance for human error. Reopening previous operative sites where bone has been allowed to regrow and fuse leads to cumbersome and time consuming steps, is messy and prevents easy access for future necessary procedures. Scarring may worsen and therefore, each re-opening of a previous operative site may prove harder, and a surgeon needs to deal with all of the layers through which scarring takes place, including the bone, meninges and over the brain surface. Furthermore, repeated re-entry requiring bony openings can lead to bone resorption and unsightly cranial defects.

Alternatively, following cranial procedures many surgeons either don't fill or cover one or multiple burr hole sites resulting in indentations and an inadequate cosmetic result, or they place superficial burr hole covers that require removal together with removal of underlying bone and scar that reform in the burr hole for future access.

In addition, currently many physicians attempt to secure a medical device to the skull with sutures and clamping screws, and then filling the burr hole with cyanoacrylate or bone cement. Securing a device with sutures and clamping screws subjects the patient to unnecessary human error. Suturing and clamping are cumbersome and time consuming steps. Filling the burr hole with cyanoacrylate or bone cement is messy and may lock the device into place, preventing easy access for future procedures.

### SUMMARY

In accordance with a first aspect of the present disclosure, there is provided a device for providing cranial access, the device comprising: a hollow shaft sized to fit within a hole extending through a skull bone, the hollow shaft having a length of at least a thickness of the skull bone; at least one connector extending laterally from a first end of the hollow shaft for abutting the skull bone around the hole; and, a plug sized to fit into the hollow shaft, the plug extending at least the length of the hollow shaft and configured to be removably secured within the hollow shaft.

Further in accordance with the first aspect, for instance, an inner surface of the hollow shaft and outer surface of the plug have corresponding threads formed thereon.

Still further in accordance with the first aspect, for instance, one of an inner surface of the hollow shaft and an outer surface of the plug has two or more radial protrusions extending therefrom, and the other of the inner surface of the hollow shaft and the outer surface of the plug has two or more corresponding L-shaped grooves formed therein.

Still further in accordance with the first aspect, for instance, a second end of the hollow shaft has an inwardly tapered outer surface to facilitate insertion of the hollow shaft into the hole.

Still further in accordance with the first aspect, for instance, the plug comprises an inflatable extension.

Still further in accordance with the first aspect, for instance, the plug has a central channel configured to receive a portion of an implanted device.

Still further in accordance with the first aspect, for instance, the implanted device is secured within the plug.

Still further in accordance with the first aspect, for instance, the plug comprises a biopsy trajectory guide.

Still further in accordance with the first aspect, for instance, the plug comprises a pressure sensor.

Still further in accordance with the first aspect, for instance, erein the plug comprises a drainage device.

Still further in accordance with the first aspect, for instance, the hollow shaft and the connector are constructed from titanium and the plug is constructed from PEEK.

Still further in accordance with the first aspect, for instance, the connector is a peripheral lip.

Still further in accordance with the first aspect, for instance, fastener holes are defined in the at least one connector.

Still further in accordance with the first aspect, for instance, bone screws are sized to be received through the fastener holes.

In accordance with a second aspect, there is provided an assembly comprising the device as above, and: a retaining ring secured within and generally concentric with the hollow shaft; and a guide defining a central channel, the guide passing through the retaining ring, the retaining ring configured to at least partially obstruct movement of the guide through the hollow shaft.

Further in accordance with the second aspect, for instance, the guide is rotatably pivoted in the hollow shaft about the retaining ring.

Still further in accordance with the second aspect, for instance, the retaining ring includes a seating surface configured to abut the guide.

Still further in accordance with the second aspect, for instance, the retaining ring includes a seating surface complementary to an at least partially spherical surface of the guide.

Still further in accordance with the second aspect, for instance, a locking ring is secured within and generically concentric with the hollow shaft, the locking ring being configured to abut the guide to hinder movement of the guide along the hollow shaft.

Still further in accordance with the second aspect, for instance, the locking ring includes a seating surface configured to abut the guide to hinder movement of the guide.

Still further in accordance with the second aspect, for instance, locking ring includes a seating surface complementary to an at least partially spherical surface of the guide.

Still further in accordance with the second aspect, for instance, a portion of the guide is sandwiched in the hollow shaft between the locking ring and retaining ring.

Still further in accordance with the second aspect, for instance, an outer surface of the guide defines first and second spherical frustums, the retaining ring having a seating surface complementary to the first spherical frustum and the locking ring have a seating surface complementary to the second spherical frustum.

Still further in accordance with the second aspect, for instance, the first and second spherical frustums are portions of a unitary continuous spherical frustum.

Still further in accordance with the second aspect, for instance, the guide is a biopsy trajectory guide.

Still further in accordance with the second aspect, for instance, an adaptor has a tubular body and defining a central channel, the central channel including a flange face facing away from the skull bone.

In accordance with a third aspect, there is provided an assembly comprising the device as above, and a tray having a tubular portion for being received in the hollow shaft, and extending outside and laterally away from the hollow shaft, the tray being disposed outside an enclosure defined by the skull bone.

In accordance with a fourth aspect, there is provided a method of closing a hole in a skull of a patient comprising inserting the as above into the hole and securing the device to the skull.

In accordance with a fifth aspect, there is provided a method of re-entering a cranial cavity of a patient after surgery comprising: inserting the device as above into a hole in the patient's skull; and removing the plug from the hollow shaft.

In accordance with a sixth aspect, there is provided a method of securing a bone flap to a skull of a patient after a craniotomy surgery wherein the bone flap, the method comprising: inserting the device as above into burr holes between the bone flap and a remainder of the skull; and securing each device to the skull by inserting screws into the skull bone through holes in the connectors.

In accordance with a seventh aspect, there is provided a device for providing cranial access, the apparatus comprising: a shaft sized to fit within a hole extending through a skull bone, the shaft having a length of at least a thickness of the skull bone; at least one connector extending laterally from a first end of the shaft for abutting the skull bone around the hole; and a cap mounted to the shaft and configured to close the hole.

In accordance with an eighth aspect, there is provided a skull linear cranial fixation plate for connecting a plate to a bone of a patient, the skull linear cranial fixation plate comprising: a bridge extending between a first fastening portion for fastening to the plate and a second fastening portion for fastening to the bone; and a member extending inferiorly from the bridge and configured to fit in a gap between the plate and the bone.

Further in accordance with the eighth aspect, for instance, the member is located equidistantly between the first fastening portion and the second fastening portion.

In accordance with a ninth aspect, there is provided a cap for a hole in a skull of a patient, comprising a cover configured to extend over the hole; and a plurality of bridges connected to the cover, each bridge including a fastening portion configured to fasten to the skull, and an extension lateral to the bridge configured to abut an inner circumference of the hole.

Further in accordance with the ninth aspect, for instance, the cover is a bone flap.

Still further in accordance with the ninth aspect the cover is in unitary construction with the plurality of bridges, and the extensions of the plurality of bridges are interconnected to form a continuous extension extending laterally from the cover into the hole.

In accordance with a tenth aspect, there is provided a cap for a hole in a skull of a patient, comprising a cover configured to extend over the hole, a plurality of fastener holes circumferentially distributed in the cover; and a shaft projecting from the cover.

One aspect provides apparatus for providing repeated cranial access comprising a hollow shaft sized to fit within a hole extending through a skull bone, the hollow shaft having a length of at least a thickness of the skull bone, a lip extending laterally form a first end of the hollow shaft for abutting the skull bone around the hole, and a plug sized to fit into the hollow shaft, the plug extending at least the length of the hollow shaft and configured to be removably secured within the hollow shaft.

The inner surface of the hollow shaft and outer surface of the plug may have corresponding threads formed thereon. One of an inner surface of the hollow shaft and an outer surface of the plug may have two or more radial protrusions extending therefrom, and the other of the inner surface of the hollow shaft and the outer surface of the plug may have two or more corresponding L-shaped grooves formed therein. A second end of the hollow shaft may have an inwardly tapered outer surface to facilitate insertion of the hollow shaft into the hole.

The plug may comprise an inflatable extension. The plug may have a central channel, or multiple channels, configured to receive a portion of an implanted device. The implanted device may be secured within the plug. The plug may comprise a biopsy trajectory guide. The plug may comprise a pressure sensor. The plug may comprise a drainage device. The hollow shaft and the lip may be constructed from titanium and the plug may be constructed from PEEK.

Another aspect provides a method of closing a hole in a skull of a patient comprising inserting an apparatus as disclosed herein into the hole and securing it to the skull.

Another aspect provides a method of re-entering a cranial cavity of a patient after surgery comprising inserting an apparatus as disclosed herein into a hole in the patient's skull, and removing the plug from the hollow shaft.

Another aspect provides a method of securing a bone flap to a skull of a patient after a craniotomy surgery wherein the bone flap was created by drilling a plurality of burr holes and interconnecting the burr holes. The method comprises inserting an apparatus as described herein into each burr hole, and securing each apparatus to the skull by inserting screws into the skull bone through holes in the lips.

Another aspect provides for an apparatus for providing repeated cranial access, the apparatus comprising: a shaft sized to fit within a hole extending through a skull bone, the shaft having a length of at least a thickness of the skull bone; and a lip extending laterally form a first end of the shaft for abutting the skull bone around the hole.

Further aspects and details of example embodiments are set forth below.

### BRIEF DESCRIPTION OF DRAWINGS

The following figures set forth embodiments in which like reference numerals denote like parts. Embodiments are illustrated by way of example and not by way of limitation in the accompanying figures.

FIG. 1A is an exploded perspective view of an exemplary skull portal device;

FIG. 1B is a top plan view of the skull portal device of FIG. 1A;

FIG. 2 is an exploded cross-sectional view of the skull portal device of FIGS. 1A-B inserted through a skull;

FIG. 3 is a perspective view of another embodiment of a plug;

FIG. 4 is a schematic perspective view of another embodiment of a skull portal device comprising the plug of FIG. 3;

FIG. 5 is an exploded cross-sectional view of the skull portal device of FIG. 4 inserted through a skull;

FIG. 6 is a schematic perspective view of another embodiment of a skull portal device comprising a plug having an inflatable extension;

FIG. 7 is an exploded perspective view of another embodiment of a skull port device, showing an exemplary cap;

FIG. 8 is a partially exploded cross-sectional view of yet another embodiment of a skull portal device inserted through the skull and featuring an adaptor;

FIG. 9 is a cross-sectional view of another embodiment of a skull portal device inserted through the skull;

FIG. 10 is cross-sectional view of another embodiment of a skull portal device inserted through the skull;

FIG. 11 is an exploded side elevation view of an embodiment of a tray assembled in an exemplary skull portal device;

FIG. 12 is a perspective view of another embodiment of a tray assembled in an exemplary skull portal device;

FIGS. 13A-B show a head of a patient during craniotomy surgery and post-surgery without and with skull portal devices of the present disclosure;

FIGS. 14A-C show an example bone plate used with skull portal devices of the present disclosure;

FIG. 15 is an exploded cross-sectional view of yet another embodiment of a skull portal device inserted through the skull;

FIG. 16 is a perspective view of an exemplary retaining ring;

FIG. 17 is a perspective view of an exemplary locking ring;

FIG. 18 is a perspective view of an exemplary guide;

FIG. 19 is a schematic cross-sectional view of the skull port device of with the locking ring attached thereto, schematically illustrating a possible range of orientations that may be adopted by the guide;

FIG. 20A is a perspective view of a bone flap connected to a skull using a plurality of skull linear cranial fixation plates;

FIG. 20B is an enlarged view of the region AA of FIG. 20A, showing an exemplary skull linear cranial fixation plate;

FIG. 20C is an exploded perspective view of the region AA of FIG. 20A; and

FIG. 21 is a perspective view of an exemplary closed cap.

### DETAILED DESCRIPTION

For simplicity and clarity of illustration, reference numerals may be repeated among the figures to indicate corresponding or analogous elements. Numerous details are set forth to provide an understanding of the examples described herein. The examples may be practiced without these details. In other instances, well-known methods, procedures, and components are not described in detail to avoid obscuring the examples described. The description is not to be considered as limited to the scope of the examples described herein.

There is a need for a portal that may provide a more rapid and permanent entry to the intracranial cavity. There may further be a need for a portal into the brain that that has a port with a centre (i.e., center) plug, which can have multiple functions and permit entry of tools and devices through which to preform operations and position devices at target sites in the brain. There may also be a need to provide a method of implanting a central plug that mitigates human error and permits safe and efficient implantation together with rapid, safe access to previous or new surgical sites. Furthermore, there may be a need for a portal that simultaneously provides cranial fixation following surgery.

The present disclosure may provide an apparatus or device, which may be referred to as a “skull portal”, “skull portal device”, having a hollow shaft or “port” that can be inserted into a hole and affixed to the skull, and a “plug” that is removably secured within the port such that the hole can be closed after surgery, and if necessary in future uses the plug can be removed to provide access to the intracranial cavity. In some embodiments, the port is sized and shaped to fit in a “burr hole” in the skull. In some embodiments, the port is sized and shaped to fit in a larger opening in the skull. In some embodiments, the apparatus according to the present disclosure can also be used to secure a bone flap to the skull (referred to as “cranial fixation”).

In some embodiments, the skull portal device with centre plug is installed and secures directly into the skull thereby creating a permanent and accessible pathway to the intracranial cavity. In some embodiments, the skull port has an outer diameter that is slightly smaller than current craniotome drill bit diameters permitting insertion through a burr hole. The center plug is capable of being mechanically unscrewed at any time in order to perform procedures in future.

Furthermore, the center plug can have multiple functions. It can fill the space preventing collapse of the burr hole, or it can house implantable devices including shunt valves, catheters, leads, and any others. In some embodiments, the skull portal device is capable of securing a medical device and being mechanically unlocked, unscrewed and disassembled to release any secured/implanted medical device. For example, in some embodiments plugs may be provided to enable securing of medical devices such as catheters or leads exiting the skull portal at appropriate angles, while remaining accessible for future procedures. In some embodiments this may be achieved by placing a device through the open central channel of the port. In other embodiments, a device may be placed through a plug that has a central channel to accommodate the device or a portion thereof (for example, an intracranial monitor, a drainage catheter, a brain stimulating lead, or other component). The device may be secured with a specifically designed plug, or the device may be secured directly to the scalp, depending on the device. In some embodiments a device such as a biopsy trajectory guide or brain tubular retractor could be secured to the port (e.g. screwed into the port, in place of a plug) in order to perform the biopsy or other procedure and integrate with currently available biopsy and brain retractor devices. The brain tubular retractor may be a plug attachment to secure to the port for work through the brain in a minimally invasive fashion.

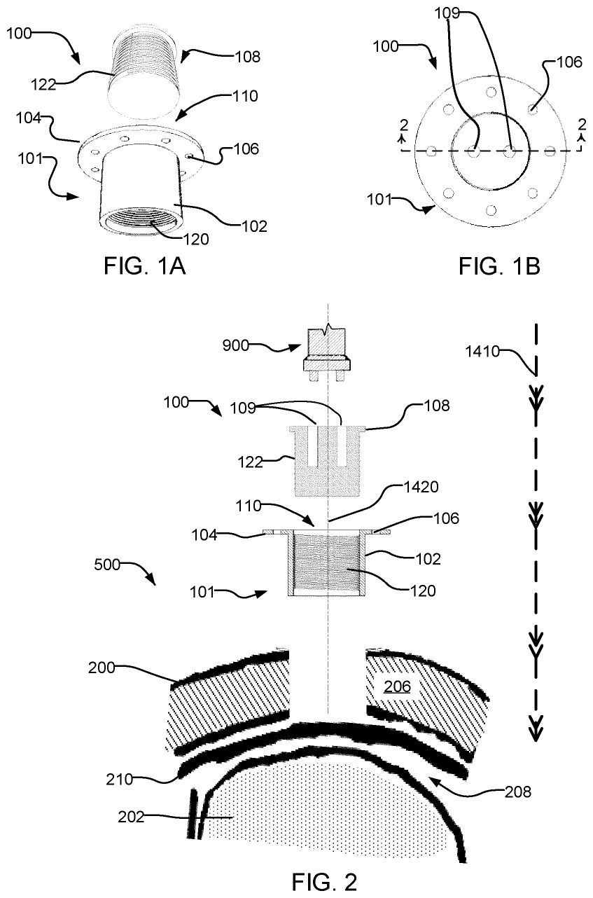

FIGS. 1A-B show an exemplary skull portal device 100. In particular, FIG. 1A is an exploded perspective view and FIG. 1B is a top plan view. FIG. 2 is an exploded cross-sectional view, including through the head 500 of a patient, of the skull portal device 100 of FIGS. 1A-B inserted through the skull 200 (part of head 500) of a patient. The cross-sectional plane of FIG. 2 is indicated in FIG. 1B by dashed line 2-2. The direction in which separate components in FIG. 2 may be brought together in assembly is schematically indicated by the series of dashed double arrows 1410. This direction may be generally oriented along an assembly axis 1420.

The skull 200 is disposed above the dura membrane 210 overlain above the brain 202. The head 500 includes skull bone 206 defining an enclosure for the brain 202. The enclosure may only be a cavity and may not be fully closed. The skull portal device 100 comprises an access port 101 that may have a shaft 102 extending through the thickness of the skull 200, but not penetrating the dura membrane 210, according to one embodiment. In this example, plug 108 is a monoblock and simply seals the opening 110.

As shown in FIGS. 1A-B and FIG. 2, the skull portal device 100 (or simply device 100) comprises an access port 101 formed by the hollow shaft 102, a.k.a., a tube, that has one or more connectors for being secured to the skull. For example, the connector is shown as being an overhanging peripheral lip 104, a.k.a., a flange, extending laterally from the hollow shaft 102. The lip 104 defines a disc (a.k.a., disk) surrounding the shaft 102, though the disc may be disrupted as well. In another embodiment, the connector(s) is defined my one or more laterally projecting tabs instead of the peripheral lip 104. The connector(s) are laid onto an outer surface of the skull. The port 101 fits in a hole in the skull 200 of the patient. The lip 104 may comprise a plurality of holes 106 for fasteners, e.g. the holes 106 may be configured to receive bone screws (not shown) to secure the access port 101 to the skull. Access port 101 may be alternatively referred to as skull port 101 (of the skull portal device 100) or simply as port 101. As referred to herein, the hollow shaft 102 extends between open ends, defining a passage, or opening 110.

The access port 101 defines the opening 110 configured to receive a plug 108. The plug 108 may be configured to be received in the access port 101 such that the plug 108 reaches the bottom of the skull bone 206, short of the dura membrane 210. The plug 108 may effectively act as a cap at both ends of the opening 110, and may be removable to maintain the patency of the access port 101. In some embodiments, the plug 108 may extended to deeper depths within the skull 200, e.g. beyond the dura membrane 210 and into the subdural space 208.

The hollow shaft 102 may comprise fastening or attachment features, e.g. disposed on an internal circumference, to couple with or secure other parts of the skull port device 100. In some embodiments, the inner surface of the hollow shaft 102 may have threads 120 that accept matching threads 122 on the outside of the plug 108, ensuring a cooperative or complementary fit and blocking inward or outward translation of the plug 108. In other embodiments, the plug 108 may be secured by other means. For example, the plug 108 may have radial protrusions that extend from an outer surface of the plug 108, and the inner surface of the hollow shaft 102 may have corresponding or complementary grooves, e.g. L-shaped grooves. In some embodiments, the inner surface of the hollow shaft 102 may have protrusions and the outer surface of the plug 108 may have grooves.

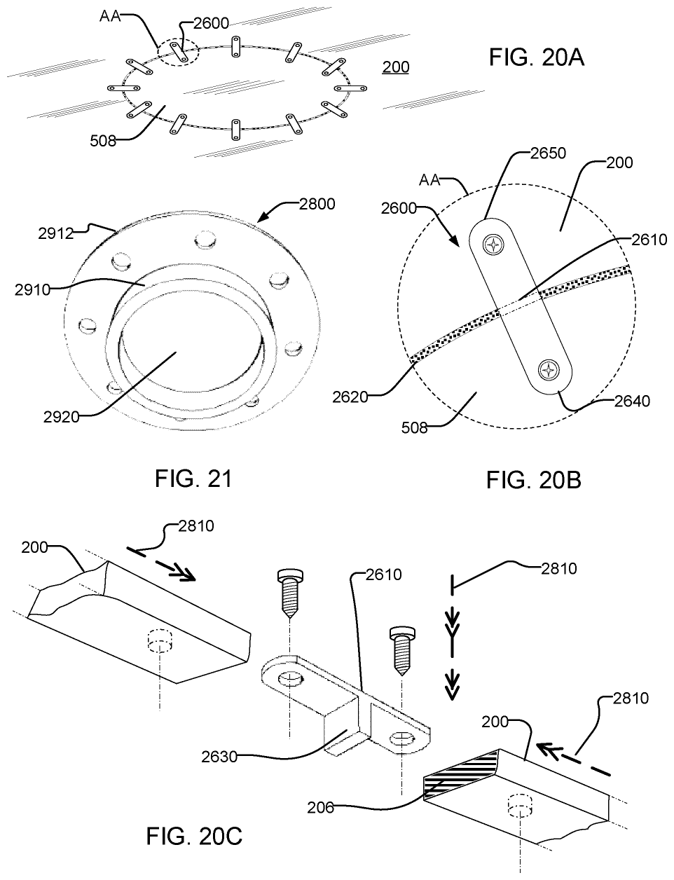

In some embodiments, the plug 108 may be secured within the port 101 using an insertion tool 900. As an illustration, the insertion tool 900 is shown in partial cross-section in FIG. 2 aligned with the skull port device 100. The top of plug 108 may have a pair of slots or holes 109 to engage with a head of the insertion tool 900 to facilitate threadable engagement of the plug 108 with the port 10, e.g. as shown in FIG. 1B and FIG. 2 the head of the insertion tool 900 may be inserted into the pair of holes 109 to insert the plug 109 into the port 101. The insertion tool 900 may be constructed from stainless steel or titanium, as an example among others, and may have a two-pronged distal surface that fits directly into the holes 109 on the top of the plug 108 permitting screwing for insertion or removal. The insertion tool 900 may comprise an elongated shaft extending between a gripping end and at an opposite end having mating features disposed thereon. The gripping end may be proximal to the bottom end of the insertion tool 900 and may comprise features to facilitate gripping, e.g. surface roughness for frictional support. The mating features may engage with features on the plug 108 to facilitate threadably engaging or pushing the plug 108 into the skull port 101. The mating features may include two or more cylindrical members (pins) extending from the top end of the insertion tool 900. For example, the mating features may include a key complementary to a keyway on the plug 108, such as in the exemplary retaining ring 1900 shown in FIGS. 19-20. Other coupling arrangements between the plug 108 and the insertion tool 900 may be used as well. For example, in some embodiments, instead of holes 109, the top of the plug 108 may have a recess configured to receive a Philips-style screwdriver, as a possibility among others.

The device 100 including the port 101 and plug 108 may be composed of various medical grade materials, such as titanium, Polyetheretherketone (a.k.a. PEEK), silicone rubber, or any other medical grade material suitable for construction and function. In some embodiments, the port 101 and plug 108 are composed of the same material. In some embodiments, the port 101 may be composed of a material to which human tissue may bond, and the plug 108 may be composed of a material that may discourage bonding with human tissue. For example, in some embodiments, the port 101 is composed of titanium, and the plug 108 is composed of PEEK. The plug 108, such as one composed of PEEK, may have smooth surfaces to discourage bonding with tissue.

The device 100 may be provided in different sizes depending on the desired application and may be patient-specific. The device 100 may be adapted for craniotomy replacement and/or reconstruction, and may include guides or outlines for creating the craniotomy bone flap to a desired size, including depth and diameter. For example, the length of the shaft 102 is selected to be at least the thickness of the skull 200 of the patient to prevent bony ingrowth into the burr hole, but the shaft 102 may be longer in certain embodiments as discussed below. The length of the shaft 102 may thus vary with the burr hole location on the skull 200 and from patient to patient, but may commonly be between 5 mm and 20 mm. However, larger and smaller shaft lengths may be possible depending on the structure of the skull 200. For example, the shaft 102 may have a length of 1-2 mm to fit in the squamous temporal bone or as large 20 mm to fit in the parietal calvarium. Furthermore, in some instances the shaft 102 may extend through the underlying dura membrane 210 and into the subdural space 208 or brain parenchyma necessitating the length of the entire device 100, shaft 102, or the plug 108 to be 8 cm or longer.

The diameter of the shaft 102 may be selected based on the diameter of the burr hole, but may commonly be between 7 mm and 16 mm. In some embodiments, the diameter of the shaft 102 may be configured to fit in a 14 mm burr hole, e.g. in various embodiments, the diameter of the shaft may be 13.8 mm, or between 13.6-13.9 mm, or other such ranges depending on the thickness of the wall of shaft 102. In some embodiments, the diameter may vary between 3 mm to 6 cm, e.g. such that the shaft 102 is suitable to fit a smaller 3 mm burr hole or serve as a larger 6 cm craniotomy bone flap replacement. The diameter of the shaft 102 may even be larger to accommodate larger craniotomy bone flaps and various skull sizes. In some embodiments, the lip 104 of the device 100 may be as thin as the titanium linear cranial fixation plates currently utilized for cranial fixation (e.g. about 0.4-0.6 mm in some embodiments), such that when placed under the skin of a patient's head the device 100 is neither unduly large nor unsightly thereby providing a generally continuous reconstruction of skull burr hole defects.

In some embodiments, e.g. when the shaft 102 is configured to fit a 14 mm burr hole, the holes 106 may each be 1 mm diameter, an outer diameter of the lip may be about 23 mm. The thickness of the wall of the shaft 102 may be 1.25 mm.

When the device 100 is used to close a burr hole in the skull of a patient, the shaft 102 is placed into the hole, such that the lip 104 sits on the outside of the skull and the port 101 is secured in place, for example by inserting self-tapping screws through the holes 106 and into the skull bone 206 around the burr hole. In some embodiments, device 100 may be inserted into the burr hole with the plug 108 already in place within the port 101. In some embodiments, the port 101 may first be secured within the burr hole, then the plug 108 secured within the port 101, for example using an insertion tool tailored to maximize the efficiency of implantation.

One important feature of the device 100 according to some embodiments is that it can extend to various depths from the skull surface (to which it is anchored), as discussed further below. Thus, the device 100 extends through the skull 200, and when necessary through the meninges (including the dura membrane 210) and into the brain. The device 100 may be manufactured with different sizes (including diameter and length) permitting access to different locations and depths depending of the functional requirement during surgery, anticipated future operations, for brain, intracranial pressure and Cerebrospinal fluid (CSF) monitoring, drainage, sampling or manipulation. Thus, depending on the selected shaft 102 length, the device 100 may provide entry to the epidural space, the subdural space 208, a subarachnoid space, the ventricular system or directly into a brain cavity (such as, but not limited to, a tumor resection cavity, a hematoma evacuation cavity, a subdural hematoma or a ventricle).

A supplementary and complementary function of the device 100 is that it provides cranial fixation and thus can be placed within any burr hole. For example, multiple devices 100 may be used to re-attach a bone flap to the skull, as discussed further below with reference to FIGS. 14A-C.

In an exemplary embodiment, the outer diameter of the shaft 102 is 13.5 mm, for a burr hole of 14 mm, the inner diameter of the shaft 102 is 12 mm, the diameter of the lip 104 is 22.5 mm, each hole 106 has diameter 1.6 mm for screws, each hole 109 has diameter 1.8 mm, and the inside of the shaft 102 has threads with a 0.75 mm pitch complementary to threads on the plug 108. In an exemplary embodiment, eight of holes 106 may be provided and two of holes 109 may be provided. In an exemplary embodiments, the two holes 109 are spaced apart by 5 mm (center-center spacing), but this may vary as a function of the tool configuration. The dimensions provided above are merely given as an example, as the shaft 102 may be dimensioned as a function of the burr hole size. Moreover, even for a burr hole of 14 mm, the dimensions of one of the devices 100 may not be exactly as provided above.

In various embodiments, the skull port device 101 may function as a brain adaptor or interface to provide multifunction access to the brain 202. For example, the plug 108 may be an insert compatible with a variety of devices configured to connect to and work with the brain 202.

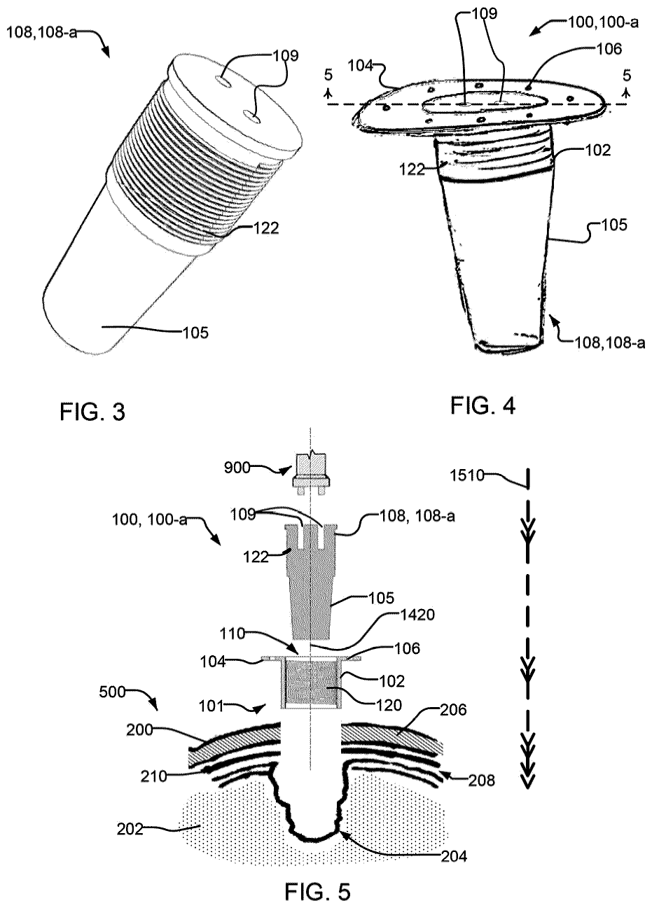

FIG. 3 is a perspective view of an embodiment of a plug 108, labelled plug 108-a. FIG. 4 is a schematic perspective view of an embodiment of a skull portal device 100, labelled skull portal device 100-a, comprising the plug 108-a of FIG. 3. FIG. 5 is an exploded cross-sectional view of the skull portal device 100-a of FIG. 4 inserted through a skull 200. The skull portal device 100-a extends into the brain 202 of the patient. The cross-sectional plane of FIG. 5 is indicated in FIG. 4 by dashed line 5-5. The direction (see assembly axis 1420) in which separate components in FIG. 5 may be brought together in assembly is schematically indicated by the series of dashed double arrows 1510.

In the example shown in FIG. 5, the brain 202 of the patient has been resected and the opening 110 of device 100-a may be used to access the resection cavity 204 of the patient. In this example, plug 108-a is a monoblock and extends beyond the shaft 102 of the device 100-a into the resection cavity 204. In some embodiments, the shaft 102 may extend into the brain 202. In various embodiments, the plug 108 may be disposed directly into the brain or the resection cavity.

In various embodiments, the plug 108-a may have a tapered lower end and may be longer than the port 101, such that the end of the plug 108-a extends below the inward tip of the port 110 and into the intracranial cavity when installed. In some embodiments, the tip of the shaft 102 (i.e., the end closest to the brain when installed) may have an inwardly tapered outer surface to facilitate insertion of the hollow shaft 102 into the burr hole. For example, the plug 108-a may have a tapered shaft 105 extending down from the bottom of the shaft 102. The shaft 105 is continuous with the plug 108-a, and may be said to be a portion of the plug 108-a. In some embodiments, the tapered shaft 105 may be constructed from a different material from the rest of the plug 108-a. For example, the tapered shaft 105 may be constructed from silicone and the rest of the plug 108-a may be constructed from titanium or PEEK.

The tapered shaft 105 of the plug 108-a may extend relatively deep in the brain 202. The plug 108-a may be configured to extend down further into the cavity due to or for tissue removal, e.g. to facilitate examination of tissue during surgery.

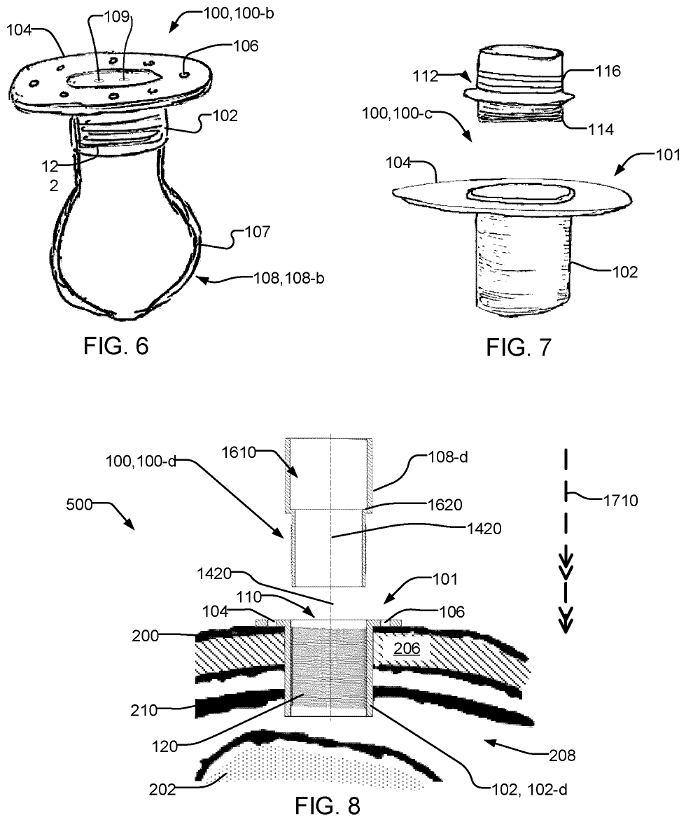

FIG. 6 is a schematic perspective view of a yet another embodiment of a skull portal device 100, labelled skull portal device 100-b, comprising a plug 108, labelled plug 108-b, having an inflatable extension 107.

In various embodiments, the plug 108 may be solid or hollow and functional or non-functional—meaning it may serve simply to fill and maintain patency of the port, or it may have different configurations that permit different functions including anchoring of different leads, wires, tubes and instruments. The plug 108 may have a solid composition, a porous composition, or one or more holes for accepting various leads, tubes and other medical instruments or devices. Certain designs of plug 108 will permit the securing or anchoring of these various devices. Furthermore, the device 100 may include one or more guides that can be inserted and attached to the port providing the ability to secure instruments including needle biopsy tools, endoscopes, laser probes, ultrasound probes, infusion devices, leads, sensors etc. as discussed below. The plug 108 and/or central channel may accommodate one or more functional inserts, e.g. for pressure monitoring and shunt placement. The plug 108, 108a-b may include a fluid reservoir, such as a drug delivery chamber, and/or a shunt valve. The plug 108 may be attached to a pump system to form (part of) a drug delivery system. Reference to embodiments of the plug 108 (and/or skull port device 100) herein is generally intended to include various embodiments of plug(s) 108-a, 108-b (and/or, respectively, skull port device(s) 100-a, 100-b).

In some embodiments, the plug 108 can be constructed of both titanium with an elongated silicone tip that extends into a tumor resection cavity, as discussed further below with reference to FIG. 10. In this configuration, the plug 108 can be removed permitting minimally invasive removal for direct re-entry to the resection cavity for the purpose of microscopic or endoscopic visualization, repeated tissue sampling or tumor removal at the time of regrowth. As another example, in some embodiments the plug 108 comprises a balloon-type extension that can be filled with a gel so as to cause its expansion to totally fill a cavity within the brain (for example, a cavity created by removal of a large tumor). Such an embodiment could be used to discourage brain matter from filling the cavity and obscuring visible and physical access to the margins of the previous surgery. In some embodiments, the balloon-type extension may facilitate post-operative balloon tamponade and hemostasis within the surgical cavity. For example, such a balloon-type extension may be composed of silicone rubber, and may be filled with a silicone gel such as the type used in breast reconstruction, etc. For example, FIG. 6 shows an embodiment with an inflatable extension 107 extending down from the bottom of the plug 108-b.

In various embodiments, the portal device 100 may be part of a kit or assembly that includes other functional elements and/or be configured to couple to other devices, e.g. tray 1100 shown in FIG. 12 and/or closed cap 2800 shown in FIG. 21. For example, FIG. 7 shows the exemplary cap 112 which could be screwed into the port 101 in order to permit attachment and use of other tools, instruments or drains during surgery. The cap 112 has a first threaded portion 114 for screwing into the port 101 and a second threaded portion 116 for engaging another device. In some embodiments, the cap 112 may be screwed into the port 101 above the plug 108 (not shown) which may include one or more openings for receiving portions of another device. The cap 112 may also be used once the plug 108 is removed. In some embodiments, the device 100 may include a trajectory guide incorporated into or coupled to the plug 108 and/or cap 112 for receiving and guiding a biopsy needle. In some embodiments, the device 100 may include a pressure sensor (e.g. an intracranial pressure monitor) incorporated into or coupled to the plug 108 for monitoring pressure inside or internal to the cranium. In some embodiments, the device 100 may include a drainage device (e.g. an external ventricular drainage catheter) incorporated into or coupled to the plug 108 and/or cap 112 for removing fluid from the cranium. The cap 112 may also serve as a skin and scalp retractor or retainer system. In some embodiments, the cap 112 may obviate the need for other self-retaining retractor systems. The cap 112 may be secured directly to the port 101 upon skin opening/re-opening to a port (upon placement thereof or to an existing port). The cap 112 may provide both a retractor function a central channel to work through directly. The cap 112 may be modified to have additional functionality such as the ability to secure instruments, scopes, light sources, and leads.

FIG. 8 is a partially exploded cross-sectional view of yet another embodiment of a skull portal device 100, labelled skull portal device 100-d, inserted through the skull 200. The skull portal device 100-d comprises a shaft 102, labelled shaft 102-d, extending into the subdural space 208, but not into the brain 202, according to one embodiment. The port 101 is shown mounted in the skull 200. The skull portal device 100-d may comprise an exemplary plug 108, not shown and removed, and an adaptor 108-d, having a tubular body defining an access conduit. The direction in which the adaptor 108-d may be brought together in assembly with the port 101 is schematically indicated by dashed double arrows 1710, which is generally oriented along the assembly axis 1420.

In some embodiments, the adaptor 108-d may complementarily engage with shaft 102-d to seal the opening 110, similar to FIG. 2 with the difference that the shaft 102-d extends into the subdural space 208.

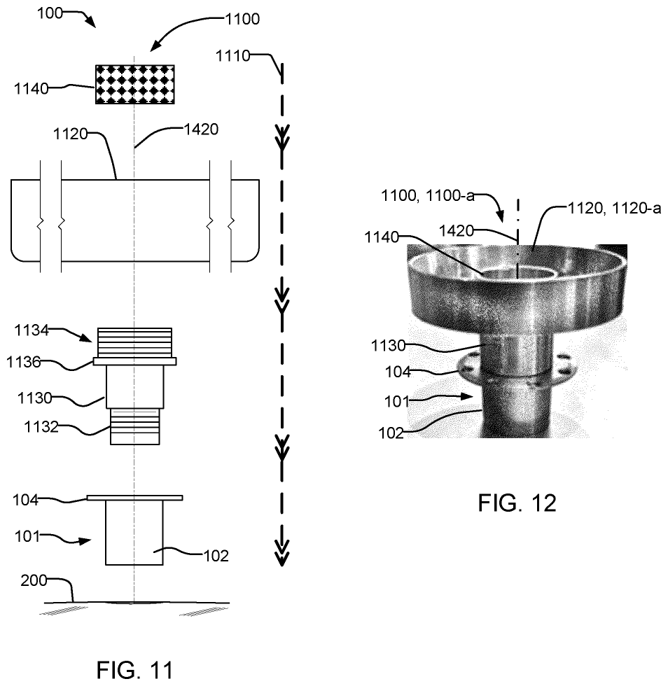

The access conduit may include a central channel passing through the adaptor 108-d. The central channel may be configured to receive a portion of an implanted device, or otherwise facilitate access via the port 101. The central channel may comprise a flange face 1620 (or shoulder) facing the skull bone 206. A flange face 1620 may be disposed inside or at an end of the access conduit of the adaptor 108-d. The flange face 1620 may support or allow resting thereon of workpieces or other components of the skull port device 100, thereby causing the access conduit 1610 to function as a scalp retainer. For example, a tray (such as tray 1100 shown in FIGS. 11-12) may be supported on the flange face 1620 for holding workpieces. The tray 1100 has a tubular portion for being received in the port 101.

The adaptor 108-d may be used to retract, or keep retracted, a scalp portion of a patient following an incision. In some embodiments, the adaptor 108-d may also facilitate insertion of a separate retractor system.

In some embodiments, the flange face 1620 may be an annular face with 10 mm internal diameter and 12 mm external diameter, corresponding to the respective diameters of the access conduit 1610.

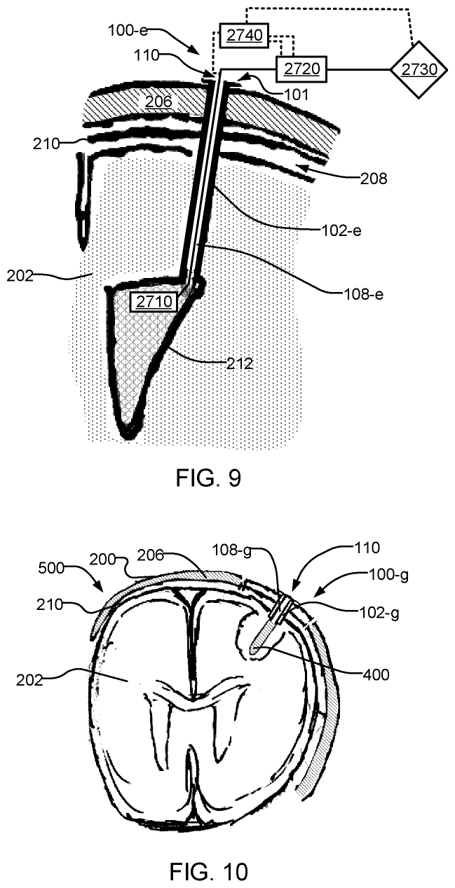

FIG. 9 is a cross-sectional view of yet another embodiment of a skull portal device 100, labelled skull portal device 100-e, inserted through the skull 200, comprising an exemplary shaft 102, labelled shaft 102-e, extending into the brain 202 of the patient. In this example, the opening 110 may be used to access a ventricle 212 of the brain 202 in order to access cerebrospinal fluid. The ventricle 212 may contain cerebrospinal fluid therein. In this example, an exemplary plug 108, labelled plug 108-e, may extend beyond the shaft 102-e of the device 100-e and into the ventricle 212. The plug 108-e may allow fluid entry, egress, sampling, infusion, removal, and/or drainage. As an alternative to the plug 108-e, the adaptor 108-d could be used in the arrangement of FIG. 9.

The plug 108-e may comprise an exemplary sensing and actuating system. The port 101 may permit a sensor 2710 to disposed in the ventricle and connected to device 2720, e.g. an data acquisition system, computer, or machine readable instructions stored in machine-readable memory and configured to operate one or more data processor(s). The device 2720 may be configured to receive input 2730. An actuator 2740 may be configured to receive input 2730, and/or signal(s) (data) from the sensor 2710 and/or the device 2720. For example, the sensor 2710 may be a pressure sensor (for measurement of intracranial pressure), temperature sensor, or concentration sensor (such as for oxygen, metabolite, drug). The actuator 2720 may be a valve configured to reduce fluid pressure by venting or removing the fluid in the ventricle. In some embodiments, the actuator 2740 may be configured for drug delivery and may include or be attached to a pump system. In various other embodiments, the sensor 2710 and actuator 2740 may include cameras, endoscopes, lights, and/or docking stations or adapters to connect components.

In various embodiments, the sensor 2710 and/or actuator 2740 may be used in conjunction with the plug 108 or may be connected thereto. For example, the plug 108 may comprise a battery or other power source to provide power to the sensor 2710 and/or actuator 2740. The sensor 2710, device 2720, and/or actuator 2740, may be wholly or partially connected to each other via wireless communication.

In various embodiments, the plug 108 (e.g. the plug 108-e) may have other functional properties including the ability to detect, measure and transmit brain electrical activity, and other physiological parameters including oxygen and metabolite (such as lactate) levels. In some embodiments, the plug 108 may be hollow or contain a hollow space for cerebrospinal fluid accumulation and thereby facilitate sampling. In some embodiments, the plug 108 may act as a reservoir to permit drug delivery to the intracranial compartment and for intracerebral drug delivery including applications such as convection-enhanced delivery. In some embodiments, the plug 108 may comprise a drainage device for all intracranial spaces including epidural, subdural, subarachnoid and ventricular. The hollow shaft and the lip may be constructed from titanium or PEEK and the plug 108 may be similarly be constructed from Titanium, PEEK, or material combinations with Titanium, PEEK, Silicone and other suitable materials. In various embodiments, the plug 108 may be suitable to transmit light, electromagnetic or other radiation, ultrasound and/or electrical fields. For example, in some embodiments, the materials such as plastic, glass, or translucent materials may be used to facilitate transmission of modalities.

In yet another embodiment of the device 100, FIG. 10 shows device 100-g inserted through skull 200 according to one embodiment. In this embodiment, shaft 102-g extends beyond the dura membrane 210, and plug 108-g, when inserted into the opening 110 extends beyond the shaft 102-g. In some embodiments the plug 108-g may have a tip 400 made of silicone or PEEK, or other resilient non-rigid material.

FIG. 11 is an exploded side elevation view of an embodiment of a tray 1100 assembled in an exemplary skull portal device 100, the tray 1100 being another possible accessory for the portal device 100. The direction in which the tray 1100 may be brought together in assembly with the port 101 is schematically indicated by dashed double arrows 1110, which is generally oriented along the assembly axis 1420. In some embodiments, the tray 1100 may removably couple and/or be part of the plug 108 and may extend outside and laterally away from the hollow shaft 102 of the port 101. The tray 1100 may be disposed outside the skull 200, i.e. outside the enclosure defined by the skull bone 206.

FIG. 12 is a perspective view of another embodiment of a tray 1100, labelled 1100-a, shown assembled in an exemplary port 101. The tray 1100-a includes a tray receptacle 1120, labelled 1120-a, comprising a circular outer sidewall defining an annular tray volume.

The tray receptacle 1120 may facilitate holding physical objects or material, e.g. instruments during a re-entry procedure. The tray receptacle 1120 may extend laterally away from the hollow shaft 102 (and/or assembly axis 1420). The outer sidewall(s) facilitate retention of objects and/or material within the receptacle. The sidewalls may define a lateral extent and depth of the tray receptacle 1120. In some embodiments, the outer sidewall may form a lateral peripheral end of the tray receptacle 1120 having a rectangular, circular, or other shape adapted to in-use requirements.

The tray 1100 may include a coupler 1130 for coupling the tray receptacle 1120 to the port 101. The coupler 1130 may include opposite fastening or attachment ends—first end 1132 and second end 1134—arranged along the assembly axis 1420 to fasten or attach to, respectively, the port 101 and the tray receptacle 1120. In some embodiments, the coupler 1130 may be integral and/or in unitary construction with the tray receptacle 1120, and thus may include (only) one free fastening or attachment end” first end 1132. In some embodiments, the first end 1132 of the coupler 1130 may be configured to threadably engage with the (inner) threads 120 of the shaft 102, and the second end 1134 may be configured to be received in the tray receptacle 1120.

The tray receptacle 1120 may include an opening for receiving the second end 1134 such that a periphery of the opening abuts a face of a flange 1136 of the coupler 1130. The flange 1136 may prevent displacement of the coupler 1130, e.g. translational displacement in at least one direction, and may generally support the tray receptacle 1120.

The second end 1134 may be engagingly received in the tray receptacle 1120 to be fastened thereto, e.g. by a complementary fastener, such as locking nut 1140 shown in FIG. 11 having surface features to provide frictional support useful for gripping and handling. The locking nut 1140 may threadably engage with the second end 1134 and, accordingly, cooperate with the (opposing) flange 1136 to sandwich a surface of the tray receptacle 1120 there-between. Thus, displacement of the tray receptacle 1120 may be hindered, e.g. it may be translationally and/or rotationally locked in-place relative to the coupler 1130 and the port 101 to which it is coupled.

In some embodiments, the tray 1100 may be used in conjunction with the adaptor 108-d. For example, the tray receptacle 1120 may rest on the flange face 1620 instead of the face of the flange 1136 of the coupler 1130.

In various embodiments, the coupler 1130 and/or locking nut 1140 may cooperatively sandwich one or more other additional or alternative accessories therebetween to substantially lock the accessories in-place relative to the port 101, rotationally and/or translationally.

In some embodiments, the locking nut 1140 may have an external diameter of about 20 mm, internal diameter about 17 mm (subject to internal threading), an axial length (parallel to assembly axis 1420) of about 16 mm, and suitable internal threading. In some embodiments, the locking nut may have an outer diameter about 17 mm.

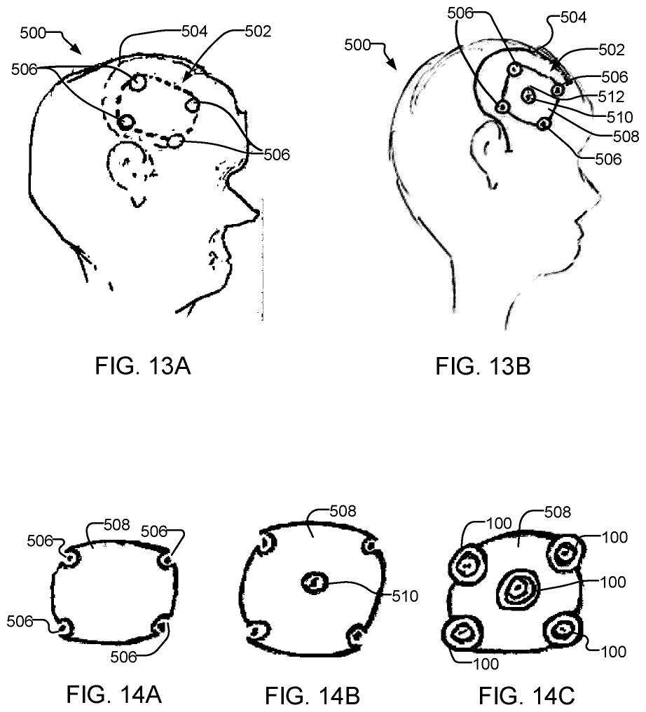

Examples of how devices according to the present disclosure may be used in craniotomy surgery are shown in FIGS. 13A-B and 14A-C. FIG. 13A shows the head 500 of a patient during brain surgery and craniotomy site 502. A question mark-shaped incision 504 (indicated by a dotted line) has been made and the skin removed. Burr holes 506 have been drilled into the skull to allow the surgeon to cut a bone flap 508, which is removed to get access to the brain. FIG. 13B shows the head 500 of a patient and craniotomy site 502 wherein the bone flap 508 has been replaced and incision 504 has been closed. In this embodiment, bone flap 508 may be fixed in place by inserting a device 100 into each of the burr holes 506, and securing the devices 100 to the skull bone 206 with screws 106, such that the devices hold the bone flap 508 in place. In this example, a central hole 510 may be made in bone flap 508 and sealed by inserting another device 100. The insertion of device 100 into the central hole 510 in the bone flap 508 permits direct access to the brain of the patient post-surgery by a small incision 512, and removal of plug 108 of device 100 if present. FIG. 14A shows a bone flap 508 that has been removed from the skull, with partial burr holes 506 around the edges thereof. FIG. 14B shows the bone flap 508 with an additional central hole 510 in bone flap 508. FIG. 14C shows the bone flap 508 fixed in place by devices 100, with another device 100 in the central hole.

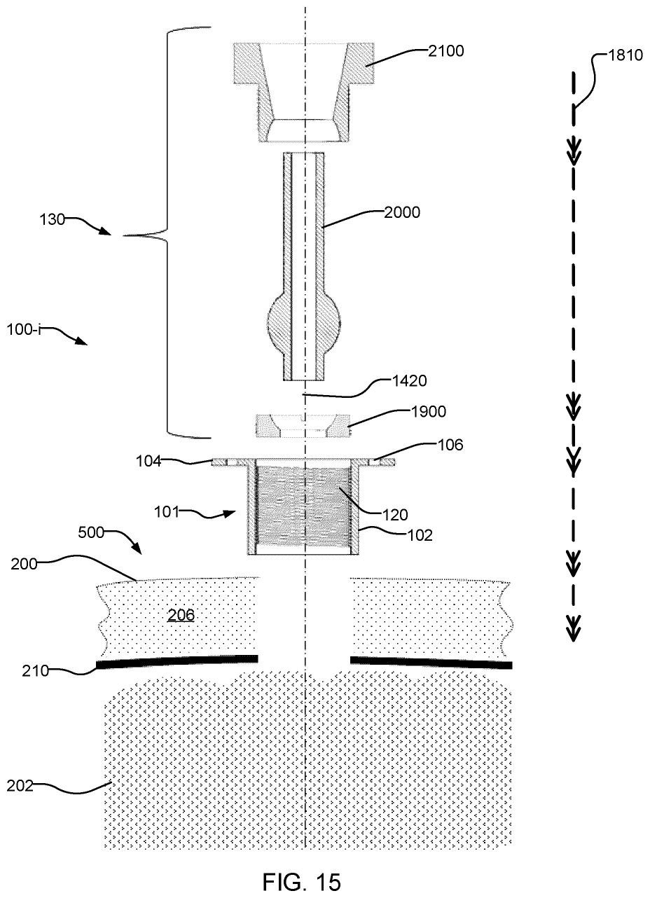

FIG. 15 is an exploded cross-sectional view of an exemplary skull port device 100, labelled skull port device 100-i, mounted in the skull 200. The direction in which separate components in FIG. 15 may be brought together in assembly is schematically indicated by the series of dashed double arrows 1810. The skull port device 100-i may include a trajectory device 130 comprising multiple parts. The trajectory device 130 may facilitate neuro-navigation and guiding an insert along a trajectory. The trajectory device 130 includes three parts: a retaining ring 1900, a guide 2000, and a locking ring 2100.

The skull port device 100-i may function as a biopsy trajectory system and the guide 2000 may be a biopsy trajectory guide configured to receive a biopsy needle in a central channel therein to extract target tissue. For example, the system may allow flexibly orienting the guide around a pivot (within a conical space) via a ball joint mechanism whereby the biopsy needle may be inserted through the trajectory device 130 inserted into the port 101. Such a system and then have sufficient degrees of freedom and mobility in order to ensure a desired trajectory. The retaining and locking rings may provide a locking mechanism permitting temporary fixation when the desired trajectory is determined and set.

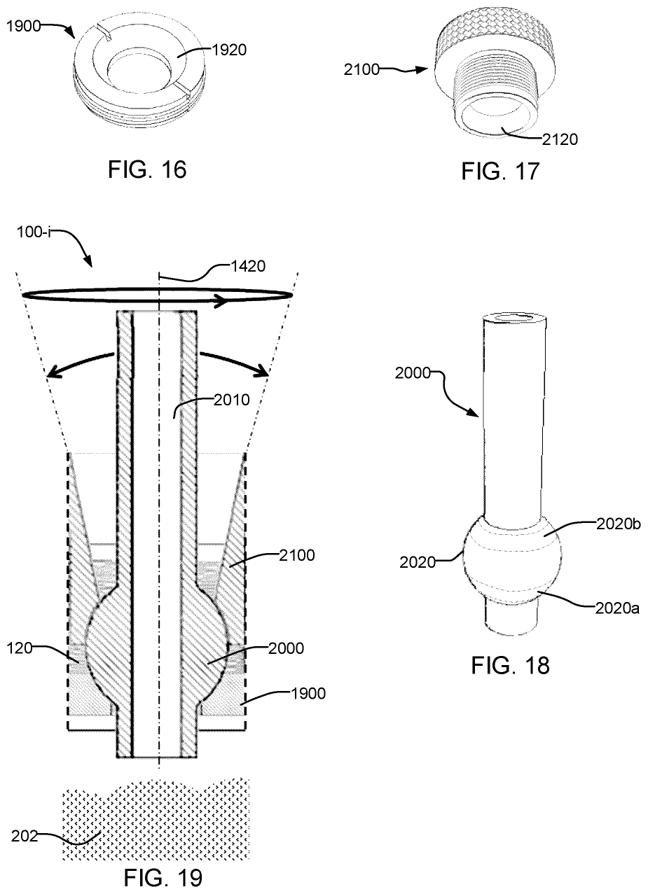

FIG. 16 is a perspective view of an exemplary retaining ring 1900. FIG. 17 is a perspective view of an exemplary locking ring 2100. FIG. 18 is a perspective view of an exemplary guide 2000. FIG. 19 is a schematic cross-sectional view of the skull port device 100-i with the locking ring 2100 attached thereto, schematically illustrating a possible range of orientations that may be adopted by the guide 2000. The orientation may be adjusted prior to engagement of the locking ring 2100 with the port 101, i.e. when the guide 2000 is resting on the retaining ring 1900 and may freely pivot thereon (via a pivoting or ball joint), and the orientation may be substantially fixed or hindered after engagement of the locking ring 2100 with the port 101. The range is indicated using thick dashed lines. In general, the guide 2000 may be oriented within a conical region having an apex at the pivot position of the guide 2000 (e.g. a central portion of the retaining ring 1900). The range of possible orientations of the guide 2000 prior to attaching the locking ring 2100 may be larger than possible orientations thereafter, due to larger allowable inclination of the guide 2000 prior to attachment of the locking ring 2100.

In reference to FIGS. 15-19, a portion of the guide 2000 may be sandwiched in the hollow shaft 102 between the locking ring 2100 and retaining ring 1900. The guide 2000 may define a central channel 2010 extending at least the length of the hollow shaft 102.

The retaining ring 1900 may be a lower ring, and the locking ring 2100 may be an upper ring. The retaining ring 1900 and the locking ring 2100 may each be secured within and generally concentric with the hollow shaft 102. The guide 2000 may pass through the retaining ring 1900.

The retaining ring 1900 may be configured to at least partially obstruct movement of the guide 2000 through the hollow shaft 102, such as translational movement therein. The locking ring 2100 may be configured to abut the guide 2000 to hinder movement of the guide 2000 along the hollow shaft 102, e.g. including preventing pivoting of the guide 2000 in the hollow shaft 102. The guide 2000 may have an outer surface tapered to at least an inner diameter of the retaining ring 1900 and, possibly separately, the locking ring 2100. Tapering may include a gradual change in outer diameter of the guide 2000 but may also include more abrupt changes to the outer diameter (like a step change).

The retaining ring 1900 may include a seating surface 1920 configured to abut the guide 2000. The locking ring 2100 may also include a seating surface 2120 configured to abut the guide 2000 to hinder movement of the guide 2000. The seating surfaces 1920, 2120 may be complementary to at least partially spherical surfaces of the guide 2000.

An outer surface of the guide 2000 may define first and second spherical frustums 2020a, 2020b. The seating surface 1920 of the retaining ring 1900 may be complementary to the first spherical frustum 2020a. The seating surface 2120 of the locking ring 2100 may be complementary to the second spherical frustum 2020b. The first and second spherical frustums 2020a, 2020b of the outer surface of the guide 2000 are portions of a unitary, continuous spherical frustum 2020. The guide 2000 may be rotatably pivoted in the hollow shaft 102 about the retaining ring 1900, e.g. via a spherical pivot.

The spherical frustum 2020 may be substantially fully spheroidal and may be configured to rest on the retaining ring 1900. Accordingly, a spherical joint may be formed between the spherical frustum 2020 and the retaining ring 1900 and/or the locking ring 2100. The guide 2000 may be constructed from a variety of possible materials. The central channel 2010 may be flexibly chosen to be of varying diameters less than an outer diameter of the spherical frustum 2020, thereby permitting placement of various instruments such as biopsy needles, leads, catheters or endoscopes. The ball movement may permit the surgeon to pick the desired trajectory to the lesion or location in question. The locking ring 2100 is then placed into the port 101 and on the ball to lock its position in place, thus fixing the desired trajectory in place for the duration necessary. The entire assembly may then be removed at the conclusion of the procedure.

An insertion tool, similar to insertion tool 900, may be used to engaged with and insert the retaining ring 1900 inside the port 101. Such an insertion tool may comprise a key configured to engage with one or more keyways on the retaining ring 1900 (such as keyways seen in FIG. 16).

In some embodiments, the locking ring 2100 may have an outer diameter of about 17 mm, and the retaining ring may have an internal diameter, opening to the hole in the skull 200, of about 6.5 mm and an external diameter of about 12 mm. The spherical frusta of locking ring 2100 and the retaining ring 1900 may be adapted to a common spheroid or ball of diameter about 9.5 mm.

FIG. 21 is a perspective view of an exemplary closed cap 2800. In some embodiments, the closed cap 2800 may facilitate closing a hole formed in the skull, e.g. the closed cap 2800 may be retained in the skull post-surgery. The closed cap 2800 may couple with the port 101 or directly with the hole in the skull 200. The closed cap 2800 may include a cap shaft 2910 sized to fit within the port or the hole of the skull, the cap shaft 2910 having a length of at least a thickness of the skull bone 206 in an embodiment. Moreover, though shown as hollow, the cap shaft 2910 may be solid or may have a plug fitted through the single open end of the cap shaft 2910. A lip 2912 may extend laterally form a first end of the cap shaft 2910 for abutting the port 101 around the opening 110 or the skull bone 206 around the hole of the skull 200. The cap shaft 2910 may form a continuous extension extending peripherally around the closed cap. A closed end 2920 of the cap shaft 2910 may form a cover to extend over and close the hole. A plurality of holes may be provided in the closed cap 2800 for attachment to the port 101 or the skull 200, e.g. via self-tapping screws. According to an embodiment, the portal device 100 includes the port 101 and the cap 2800, but no plug 108.

Thus, the cap 2800 forms a cover from which projects shaft 2910, the shafts 2910 for instance projecting perpendicularly from a plane of the cover. The cover therefore defines a closed end 2920 that may be an embodiment of the plate (bone flap 508). The cover may also define the lip 2912 or flange or like connector(s). In such embodiments, the cover is in unitary construction. The cap 2800 has the shaft 2910 selected in dimension as a function of the burr hole or skull opening. The shaft 2910 therefore serves a cetering function by causing the fastener holes circumferentially distributed in the lip 2912 or equivalent connector(s) (e.g., tabs) to be spaced sufficiently from the periphery of the skull opening. The shaft 2910 may be concentric with an imaginary circle passing through the center of the fastener holes, as a possibility.

FIG. 20A is a perspective view of a bone flap 508 connected to the skull 200 using a plurality of skull linear cranial fixation plates 2600. FIG. 20B is an enlarged view of the region AA of FIG. 20A, showing an exemplary skull linear cranial fixation plate 2600. FIG. 20C is an exploded perspective view of the region AA of FIG. 20A. The direction in which separate components in FIG. 20C may be brought together in assembly is schematically indicated by the series of dashed double arrows 2610. In reference to FIGS. 20A-C, the skull linear cranial fixation plates 2600 connect a plate, e.g. a bone flap 508 or a plate made of another biologically compatible material, to the skull bone 206. In some embodiments, the bone flap 508 may be connected to other bone flaps or pieces. The linear cranial fixation plates connect the bone flap 508 (or plate) and skull 200 via a dual fastener arrangement wherein a first fastening portion 2640 is configured to fasten to the bone flap 508 and a second fastening portion 2650 is configured to fasten to the skull bone 206. A bridge 2610 extends between the first and second fastening portions 2640, 2650 over a gap 2620 defined between the bone flap 508 and the skull bone 206. The gap 2620 is a region of the skull 200 wherein the skull bone 206 has been removed and is missing, e.g. due to cutting of skull bone 206 during a craniotomy procedure.

If the linear cranial fixation plate is fastened excessively close to the bone gap 2620 on either the bone flap 508 or the skull 200, the material supporting the fastening may chip or break and cause the linear cranial fixation plate to fail. It may therefore be necessary to properly align the linear cranial fixation plate 2600 prior to fastening. Furthermore, such alignment may need to be maintained by a surgeon, possibly for a relatively prolonged period of time and possibly by hand.

A member 2630, which may also be referred to as a ridge, a catch, etc or extends inferiorly from the bridge 2610 and is configured to fit in the gap 2620. Stated differently, the member 2630 projects from a plane of the bridge 2610. The member 2630 thus allows self-alignment and/or self-positioning of the linear cranial fixation plate between the bone flap 508 and skull 200, such as into a gap between them. For example, the member 2630 may be positioned equidistant between the two fastening portions 2640, 2650 to ensure the linear cranial fixation plate 2600 support providing the connection between the bone flap 508 and the skull 200 is equally distant, or at a desired distance, from the bone gap 2620 on either side of the connection (i.e. from the bone flap 508 and the skull 200). The self-alignment of the skull linear cranial fixation plate 2600 facilitates maintaining a relatively stationary alignment between the bone flap 508 and the skull 200.

Although the embodiments have been described in detail, it should be understood that various changes, substitutions and alterations can be made herein. Moreover, the scope of the present application is not intended to be limited to the particular embodiments of the process, machine, manufacture, composition of matter, means, methods and steps described in the specification. As can be understood, the examples described above and illustrated are intended to be exemplary only.

As will be apparent to those skilled in the art in light of the foregoing disclosure, many alterations and modifications are possible to the methods and systems described herein. While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as may reasonably be inferred by one skilled in the art. The scope of the claims should not be limited by the embodiments set forth in the examples, but should be given the broadest interpretation consistent with the foregoing disclosure.

The present disclosure may be embodied in other specific forms without departing from its spirit or essential characteristics. The described embodiments are to be considered in all respects only as illustrative and not restrictive.

1. A device for providing cranial access, the device comprising:
a hollow shaft sized to fit within a hole extending through a skull bone, the hollow shaft having a length of at least a thickness of the skull bone;
at least one connector extending laterally from a first end of the hollow shaft for abutting the skull bone around the hole; and,
a plug sized to fit into the hollow shaft, the plug extending at least the length of the hollow shaft and configured to be removably secured within the hollow shaft.

2. The device of claim 1 wherein an inner surface of the hollow shaft and outer surface of the plug have corresponding threads formed thereon.

3. The device of claim 1 wherein one of an inner surface of the hollow shaft and an outer surface of the plug has two or more radial protrusions extending therefrom, and the other of the inner surface of the hollow shaft and the outer surface of the plug has two or more corresponding L-shaped grooves formed therein.

4. The device of claim 1 wherein a second end of the hollow shaft has an inwardly tapered outer surface to facilitate insertion of the hollow shaft into the hole.

5. The device of claim 1 wherein the plug comprises an inflatable extension.

6. The device of claim 1 wherein the plug has a central channel configured to receive a portion of an implanted device.

7. The device of claim 6 wherein the implanted device is secured within the plug.

8. The device of claim 1 wherein the plug includes a biopsy trajectory guide.

9. The device of claim 1 wherein the plug includes a pressure sensor.

10. The device of claim 1 wherein the plug includes a drainage device.

11. The device of claim 1 wherein the hollow shaft and the connector are constructed from titanium and the plug is constructed from PEEK.

12. The device of claim 1 wherein the connector is a peripheral lip.

13. The device of claim 1 wherein fastener holes are defined in the at least one connector.

14. The device according to claim 13, further including bone screws sized to be received through the fastener holes.

15. An assembly comprising the device of claim 1, and:
a retaining ring secured within and generally concentric with the hollow shaft; and
a guide defining a central channel, the guide passing through the retaining ring, the retaining ring configured to at least partially obstruct movement of the guide through the hollow shaft.

16. The assembly of claim 15, wherein the guide is rotatably pivoted in the hollow shaft about the retaining ring.

17. The assembly of claim 15, wherein the retaining ring includes a seating surface configured to abut the guide.

18. The assembly of claim 15, wherein the retaining ring includes a seating surface complementary to an at least partially spherical surface of the guide.

19. The assembly of claim 15, comprising
a locking ring secured within and generically concentric with the hollow shaft, the locking ring being configured to abut the guide to hinder movement of the guide along the hollow shaft.

20. The assembly of claim 19, wherein the locking ring includes a seating surface configured to abut the guide to hinder movement of the guide.

21.-37. (canceled)
